(19) United States
(12) Patent Application Publication
(10) Pub. No.: US 2011/0208268 A1
(43) Pub. Date: Aug. 25, 2011

(54) SYSTEM AND METHOD FOR POWER-ON-RESET DETECTION AND DECODING

(76) Inventor: Timothy R. Brown, Maple Grove, MN (US)

(21) Appl. No.: 12/712,945

(22) Filed: Feb. 25, 2010

(51) Int. Cl. A61N 1/08

(52) U.S. Cl. 607/60

(57) ABSTRACT

Systems, methods and devices are provided for automatically detecting and classifying power-on-reset (POR) events occurring in medical devices. Data regarding the operating parameters of the medical device is communicated to a remote server where a POR detection algorithm is operated to detect whether a POR event occurred in the medical device. Upon detection of a POR event, the remote server determines a severity rating of the POR event, a basis of the POR event, and/or the time of the POR event, where this information is associated with the POR event itself. An automated alert message is then generated and communicated to at least one of a patient, a clinician, a care giver or another device with the information about the POR event.

### TECHNICAL FIELD

This disclosure relates generally to medical devices and more particularly to a system and method for detecting and decoding Power-On-Reset (POR) events in implantable medical devices (IMDs).

### BACKGROUND

There are events both internal and external to an implantable medical device (IMD) that can disrupt normal IMD functionality. For example, internal events occurring within the IMD, such as a battery malfunction, memory cell retention error, or therapy malfunction (e.g., pacing malfunction) are events that, when detected, can disrupt normal IMD functionality and result in a Power-On-Reset (POR) to occur within the IMD. POR is a function performed in an IMD to reset the IMD to known software and/or hardware states of operation in response to conditions or events experienced by the IMD that disrupt its normal functionality. Aside from internal events occurring within the IMD that can result in POR, external events, such as transthorasic shock near the IMD, magnetic resonance imaging (MRI) signals, or other external signals (e.g., from electromagnetic signals from theft detectors or solar radiation), can also result in a POR occurring in an IMD. When such conditions or events disrupt normal IMD functionality, it is desirable to restore the IMD to a known error-free state of operation through use of a POR operation.

Conventionally, upon the occurrence of a POR event in an IMD, the IMD is often configured to issue an audible alert in connection with the POR to notify the patient that a POR has occurred. Even though the IMD may be functioning properly, when POR operations occur frequently, these alerts can cause the patient to question the integrity and functionality of their specific IMD, which historically has led to patients frequently contacting their physicians or the manufacturer of the IMD to ensure that the IMD is functioning properly.

### SUMMARY

Systems, methods and devices are described herein for automatically detecting and decoding Power-On-Reset (POR) events in implantable medical devices (IMDs). Information and data relating to the operating parameters of the IMD is communicated to a remote server, where a POR detection algorithm is operating to detect whether a POR event occurred in the IMD. Upon detection of a POR event, the remote server determines a severity rating of the POR event, a basis of the POR event, and/or the time of the POR event, where this determined information is associated with the POR event itself. An automated alert message is then generated and communicated to at least one of a patient, a clinician, a care giver or another device with the information about the POR event. This enables substantially real-time alerts to be generated in an automated manner with a prompt notification of the reason for the POR event, thereby improving patient satisfaction and understanding of the operation of the IMD.

### DRAWINGS

The above-mentioned features and objects of the present disclosure will become more apparent with reference to the following description taken in conjunction with the accompanying drawings wherein like reference numerals denote like elements and in which:

FIG. 1 illustrates components of a system including an implantable medical device in accordance with one or more embodiments of the present disclosure.

FIG. 2 illustrates components of an implantable medical device in accordance with one or more embodiments of the present disclosure.

FIG. 3 is a schematic block diagram illustrating the various system components including an implantable medical device, an external device and a remote server in accordance with one or more embodiments of the present disclosure.

FIG. 4 is an operational flow diagram illustrating an automated process for detecting power-on-reset (POR) events in an implantable medical device in a patient in accordance with one or more embodiments of the present disclosure.

### DETAILED DESCRIPTION

In the following detailed description of embodiments of the present disclosure, reference is made to the accompanying drawings in which like references indicate similar elements, and in which is shown by way of illustration specific embodiments in which the present disclosure may be practiced. These embodiments are described in sufficient detail to enable those skilled in the art to practice the present disclosure, and it is to be understood that other embodiments may be utilized and that logical, mechanical, electrical, functional, and other changes may be made without departing from the scope of the present disclosure. The following detailed description is, therefore, not to be taken in a limiting sense, and the scope of the present disclosure is defined only by the appended claims.

Various embodiments described within this disclosure will be directed to systems, methods and devices for detecting and decoding Power-On-Reset (POR) events in implantable medical devices (IMDs).

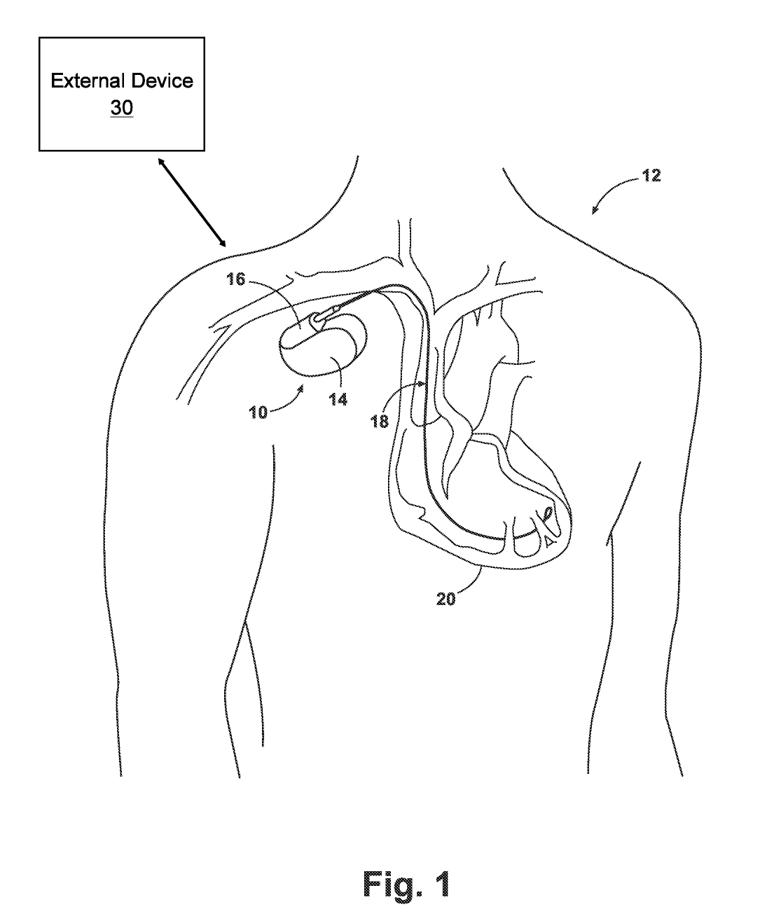

As illustrated in FIG. 1, IMD 10 is described in various embodiments as comprising an implantable medical device that is implantable within a patient 12 including sensing capabilities for monitoring physiological conditions and may additionally or alternatively include alert and therapy delivery capabilities. An IMD 10 in which the invention is implemented may be primarily intended for detecting any type of medical condition. For example, the IMD 10 may comprise any type of implanted device or subcutaneous device including, but not limited to implantable pulse generators (IPGs) (e.g., cardiac pacemakers), implantable cardioverter-defibrillators (ICDs), implantable combination pacemaker-cardioverter-defibrillator (PCDs), implantable brain stimulators, implantable gastric system stimulators, implantable nerve stimulators or muscle stimulators, implantable lower colon stimulators, implantable drug or beneficial agent dispensers or pumps, implantable cardiac signal loops or other types of recorders or monitors, implantable gene therapy delivery devices, implantable incontinence prevention or monitoring devices, implantable insulin pumps or monitoring devices, or any other type of implantable medical device. In some embodiments, the IMD 10 may comprise a similar monitoring, diagnostic, or therapeutic medical device that is worn by or otherwise attached to the exterior of the patient 12.

A wide variety of IMDs 10 have been developed in order to monitor patient conditions and deliver therapy to the patient. By way of example only, FIG. 1 provides a perspective view of an IMD 10 implanted within a human body 12 in which one or more embodiments of the invention may be implemented. IMD 10 of FIG. 1 comprises a hermetically sealed housing 14 (or “can”) and connector header or block module 16 for coupling IMD 10 to electrical leads and other physiological sensors arranged within body 12, such as pacing and sensing leads 18 connected to portions of a heart 20 for delivery of pacing pulses to a patient's heart 20 and sensing of heart 20 conditions in a manner well known in the art. For example, such leads may enter at an end of header block 16 and be physically and electrically connected to conductive receptacles, terminals, or other conductive features located within header block 16. IMD 10 may be adapted to be implanted subcutaneously in the body of a patient such that it becomes encased within body tissue and fluids, which may include epidermal layers, subcutaneous fat layers, and/or muscle layers.

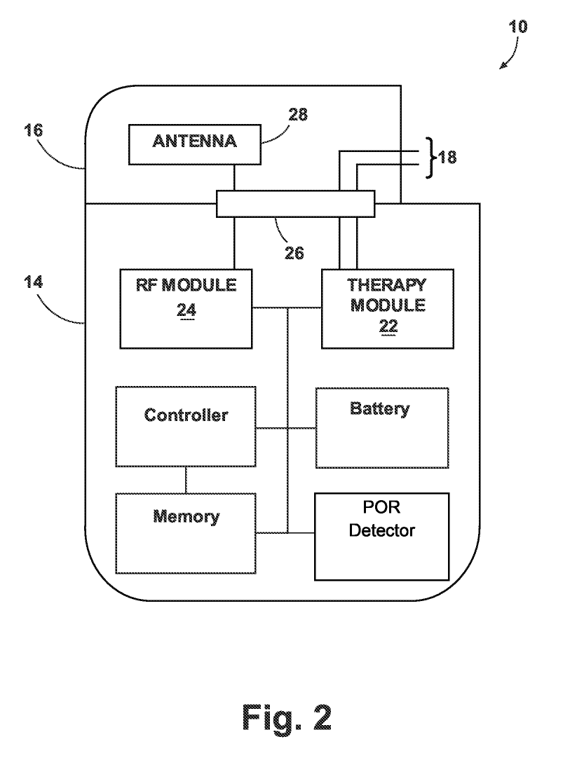

Housing 14 may contain a number of functional elements, components, and features, including (without limitation): a battery; a high voltage output capacitor; integrated circuit (“IC”) devices; a processor; memory elements; a therapy module or circuitry; an RF module or circuitry; and an antenna matching circuit. During the manufacturing process, electrical connections are established between components located within housing 14 and elements located outside of housing 14, such as within header block 16. FIG. 2 is a simplified schematic representation of an IMD 10 and several functional elements associated therewith. IMD 10 may include a header block 16 coupled to housing 14, a therapy module 22 contained within housing 14, and an RF module 24 contained within housing 14. A feedthrough 26 structure may bridge the transition between housing 14 and header block 16. Antenna 28 is coupled to RF module 24 to facilitate RF telemetry between IMD 10 and an external device 30. In practice, IMD 10 will also include a number of conventional components and features necessary to support the functionality of IMD 10 as known in the art. Such conventional elements will not be described herein.

It is understood that the particular configuration illustrated in FIGS. 1 and 2 is for purposes of illustration only and IMD 10 may comprise any type of medical device, implantable or externally worn on the body 12 of a patient. The invention may also be implemented in any medical devices that may be used for monitoring of a patient for detecting conditions at a variety of physical locations, such as a patient's home, a physician's office, a hospital or a treating emergency technician.

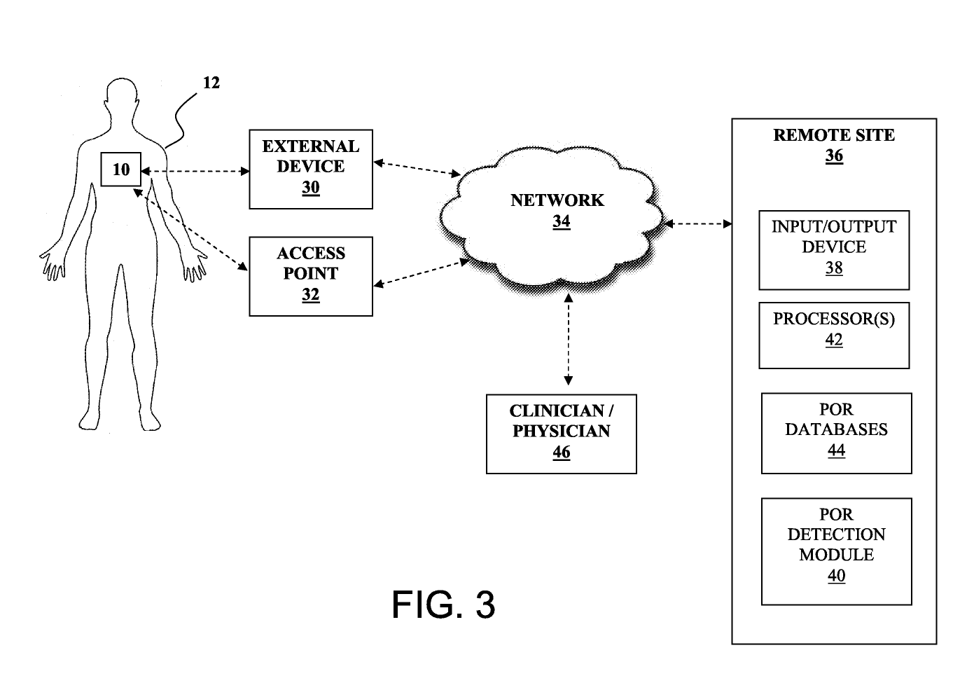

In one or more embodiments, external device 30 that communicates with IMD 10 may comprise an external medical device, a programming device, a remote telemetry station, a physician-activated device, a patient-activated device, a mobile handheld unit (e.g., mobile phone, PDA, etc.), a personal computer, an in-home monitoring device, a patient-wearable device, a display device or any other type of device capable of sending and receiving signals to and from IMD 10. In one or more embodiments, external device 30 may comprise an in-home monitoring device, such as the Medtronic CareLink® Network monitor, that collects information from IMD 10 and communicates such information to a remote site 36 (e.g., a remote server, remote clinicians, remote devices or the like) through a network connection 34. The network connection 34 may comprise the Internet, a local computer network, phone lines, wireless networks, cellular network or other data communication pathways, as illustrated in FIG. 3. In one or more embodiments, the remote site 36 may comprise a remote server (such as a remote server of the CareLink® Network available from Medtronic, Inc. of Minneapolis, Minn.), where the data may be stored or from which a clinician or another individual may access or review the data. Carelink is a registered trademark of Medtronic, Inc. of Minneapolis, Minn. For ease of description, remote site 36 will hereafter be referred to as remote server 36.

In one or more embodiments, IMD 10 may transmit data and information to and receive information from external device 30 related to the operation of IMD 10, such as the operating parameters of IMD 10. Upon receiving such information, external device 30 may upload the received information to the remote server 36. In some embodiments, the information transmitted from IMD 10 may be accessed directly by directly interacting with external device 30. External device 30 may further include a display, a speaker and other audio/visual indicators from which information and data may be provided to the patient, a clinician or another individual (e.g., information related to the operation of IMD 10, alert messages, etc.). In one or more embodiments, external device 30 may comprise a personal computer, mobile phone or other programmable electronic device having a software program installed thereon configured for receiving data from IMD 10, processing such data and/or further communicating such data to a remote location or clinician for further analysis and/or processing.

In one or more embodiments, information may also be communicated from IMD 10 to remote site 36 without passing through external device 30, such as by communicating information through an access point 32 (e.g., a wireless access point) in communication with IMD 10 that serves as a relay point for communicated information.

Communication between IMD 10 and external device 30 or access point 32 can occur via telemetry, such as a long-distance telemetry system through an RF telemetry module that facilitates wireless data transfer between IMD 10 and external device 30 or access point 32. The devices may be configured to perform any type of wireless communication, such as but not limited to radio frequency (RF) signals, infrared (IR) frequency signals, or other electromagnetic signals. Any of a variety of modulation techniques may be used to modulate data on a respective electromagnetic carrier wave. Alternatively, sound waves may be used for communicating data or the patient's tissue may be as the transmission medium for communicating with a programmer positioned on the patients skin. Other types of wired communications may also occur when IMD 10 is alternatively configured as an external device or contains wired communication channels that extend from within the patient to points outside of the patient.

In one or more embodiments, IMD 10 may include internal hardware (e.g, circuitry) or programmed software routines (e.g., firmware, stored or downloaded software program modules) that monitor the operation of IMD to detect if and when a Power-On-Reset (POR) event occurs in IMD 10. A POR is a function performed in IMD 10 to reset the IMD 10 to known software and/or hardware states of operation in response to conditions or events experienced by IMD 10 that disrupt its normal functionality. The types of events that can disrupt normal IMD 10 functionality include internal events occurring within IMD 10, such as a battery malfunction, memory cell retention error, or therapy malfunction (e.g., pacing malfunction) or external events, such as transthorasic shock near the IMD, magnetic resonance imaging (MRI) signals, or other external signals (e.g., from electromagnetic signals from theft detectors or solar radiation). When such conditions or events disrupt normal IMD 10 functionality, it is desirable to restore IMD 10 to a known error-free state of operation through use of a POR operation.

Conventionally, upon the occurrence of a POR event in an IMD, IMD's were often configured to issue an audible alert in connection with the occurrence of the POR to notify the patient that a POR had occurred. Even though the IMD may have been functioning properly, when POR operations occur frequently, these alerts traditionally caused patients to question the integrity and functionality of their specific IMD, which historically has led to patients frequently contacting their physicians or the manufacturer of the IMD to ensure that the IMD is functioning properly. In one or more embodiments, IMD 10 may similarly include an audio device (e.g., a speaker) that issues an audible alert in connection with the POR.

In one or more embodiments, when a POR occurs, IMD 10 is configured to communicate information relating to the occurrence of the POR event to remove server 36, such as a notification that the POR occurred and any other operational parameters of IMD 10 that would be indicative of or relevant to the POR event. Such operational parameters may include, but are not limited to, the time and date of the POR, the cause of the POR, the operation of IMD 10 when the POR occurred, parity events, sensed or stored information in the memory of IMD 10, etc.

In one or more embodiments, remote server 36 is configured to analyze the information it receives from IMD 10 to determine if a POR occurred. Upon detection of a POR, further information relating to the POR is determined, such as, but not limited to, the time and date of the POR event, a reason or basis for the POR event, and/or an associated severity rating of the POR. With respect to the severity rating of the POR, in one or more embodiments, the POR may be categorized or ranked based on the reason for the occurrence of the POR. Depending upon the categorized or ranked severity rating, determinations can be made for recommended actions. For example, the POR may be categorized with 1) a high or urgent severity rating that may require urgent actions (e.g., IMD 10 replacement or repair), 2) a medium severity rating that may require IMD 10 operations to be monitored or further investigated to ensure IMD 10 is functioning properly, or 3) a low severity rating in which no actions are required. There are some low severity “soft errors,” such as when a memory cell is changed and detected by an on-going firmware memory integrity check, where IMD 10 is still functioning properly and no actions are required.

Referring further to FIG. 3, in one or more embodiments, remote server 36 includes in an input/output device 38 for communicating with IMD 10 and/or external device 30 through network 34, where such input/output device 38 may comprise any data communication device capable of communicating data across network 34, depending upon the type of network 34 employed, as well known to those skilled in the art. Remover server 36 further includes a POR detection module 40 containing instructions stored in a memory of remote server 36 to provide functionality as described herein for the POR detection algorithm mechanisms of remote server 36. In one or more embodiments, remote server 36 includes at least one processor 42 or controller that is configured to operate control algorithms and the POR detection module 40 algorithm stored in memory of the remote server 36. Processor 42 may be implemented with any type of microprocessor, digital signal processor, application specific integrated circuit (ASIC), field programmable gate array (FPGA) or other integrated or discrete logic circuitry programmed or otherwise configured to provide functionality as described herein. Processor 42 executes instructions stored in memory for the POR detection module 40, where instructions provided to processor 42 may be executed in any manner, using any data structures, architecture, programming language and/or other techniques. The memory of remote server may comprise any storage medium capable of maintaining digital data and instructions for POR detection module 40 and other operation of remote server such as a static or dynamic random access memory (RAM), read-only memory (ROM), non-volatile random access memory (NVRAM), electrically erasable programmable read-only memory (EEPROM), flash memory, or any other electronic, magnetic, optical or other storage medium.

In one or more embodiments, remote server 36 may further include POR database(s) 44 that include stored relational information that may be retrieved and used for comparison purposes in detecting and classifying the severity of POR events.

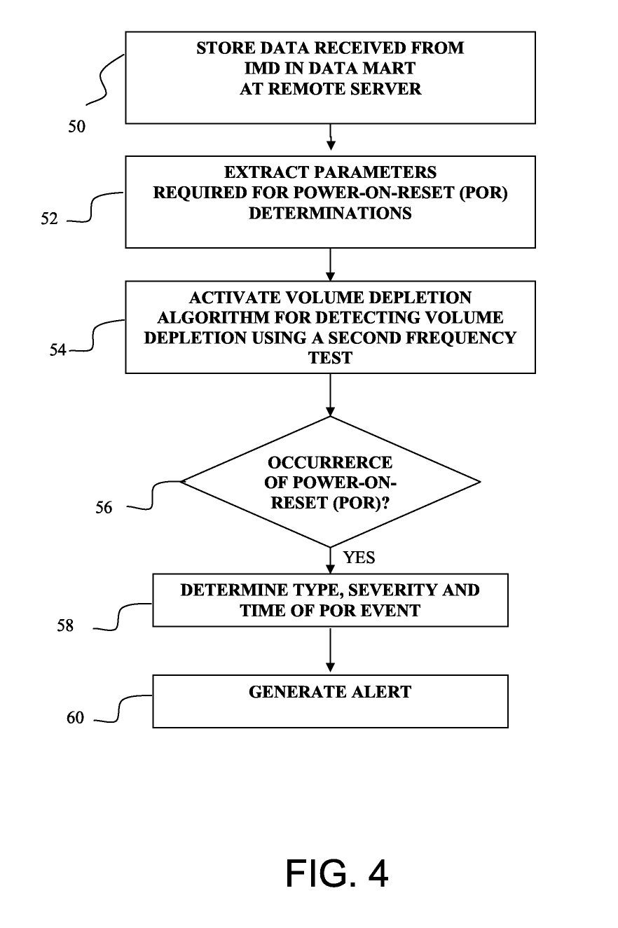

Referring now to FIG. 4, an operational flow diagram is illustrated in accordance with one or more embodiments of the present disclosure for the algorithm of POR detection module 40 for detecting whether a POR has occurred and, if so, identifying and classifying the POR. In operation 50, information that is communicated from IMD 10 to remote server 36 is stored in a data mart in the memory of remote server 36. As described above, this information may include the fact that the POR occurred and any other operational parameters that would be indicative of or relevant to the POR event in IMD 10. The parameters required by POR detection module 40 to make the necessary determinations are retrieved from memory in operation 52, where these retrieved parameters are then decoded and/or analyzed in operation 54 to determined whether a POR occurred in the IMD 10. The particular parameters stored, retrieved and analyzed may vary depending upon the type of IMD 10 and the type of POR event. For example, the retrieved parameters may include the indication of a POR event and certain parity events, where the parameters are decoded to determine the extent (i.e., severity rating) of the POR. If it is determined that a POR in fact occurred (in operation 56), then information relating to the POR, such as, but not limited to, the time and date of the POR event, a reason for the POR event (e.g., type of POR event), and/or an associated severity rating of the POR is generated in operation 58. In one or more embodiments, the reason for the POR event may include a simple or layman's reason for why the POR event occurred, such that it would be easy almost any individual, including the patient, to understand the basis of the POR.

In one or more embodiments, an alert message may be generated in operation 60 to provide a notification of the POR determination, the type of the POR, the reasoning for the POR, and/or the time and date of the POR event. This alert message may be communicated the patient, a clinician 46 (see FIG. 3) or another individual, the external device 30 or another remote device. By being provided with such notification, patients 12 having IMD 10 can be provided with an immediate analysis of the POR and a device integrity communication. This immediate analysis can calm the fears and worries of patients by assuring them that their IMD 10 is functioning properly and provide them with the reasoning for the POR of their IMD 10. Alternatively, if a POR is of an urgent nature, this immediate notification to the patient 12 can allow the patient to seek more rapid attention for their IMD 10. Furthermore, by being provided with the time of the POR event and the layman's reason for why the POR event occurred, this enables the patient to link the POR with external events that are known causes of inducing PORs, such as radiation therapy, a MRI, or their surrounding environment. Along these same lines, the clinician 46 is also provided with an immediate notification that allows the clinician to take appropriate actions, if required.

The POR module 40 operating at remote server 36 provides for an automated manner of immediately identifying the rationale for a POR 10 in a consistent and uniform basis, thereby improving clinician/physician determinations for required actions and reducing the need for constant clinician-patient interaction. In the past with conventional POR events occurring in IMDs, patients and physicians needed to report the occurrences of POR events to the manufacturer or servicer of the IMDs, who in turn would utilize customer service groups to analyze the data surrounding the POR events to attempt to determine why the POR occurred. This required personnel training for the customer service groups and also led to inconsistent determinations based on the particular personnel analyzing the results, thereby making the results prone to misinterpretation. The customer service groups would then report back to the physicians and/or patients, thereby leading to both inconsistent results and an entirely inefficient process. By utilizing the automated procedures employed by the POR module 40, consistent alert messages can be generated, thereby improving the accuracy of the determination of the functionality of the IMD 10. Furthermore, information relating to the past operational history of the IMD 10 can also be analyzed in or more embodiments, yet even further improving the accuracy of the determinations of the functionality of the IMD 10.

Following in Table 1 below is a listing of representative types of POR events along with possible examples of their severity ratings that may be assigned in accordance with one or more embodiments for certain types of IMD 10:

TABLE 1

| Type of POR | Severity Rating |
|---|---|
| Watchdog POR (pulsed) | Medium |
| Watchdog POR (held) | High |
| Atrial & Ventricle Rate Limit | Medium |
| Stack Overflow/Underflow | Medium |
| Memory Error | Low/Medium |
| Software Interrupt (SWI) Error | Low |
| VDD Glitch | High |
| VDD Monitor | High |
| Vreg Glitch | High |
| Vreg Monitor | High |
| Illegal Stop Key | Medium |
| Critical RAM Parity Error | Low |
| Write to Locked RAM | Low |
| Write to ROM | Medium |
| Write or Read to Illegal Address | Medium |
| Illegal Pacing Mode | Medium |
| Non-Maskable Interrupt | Medium |
| Event Buffer Overflow | Medium |
| Parity Check | Low/Medium |
| Illegal Op Code | Medium |
| CPU Error | Medium |
| EEPROM POR | High |
| Firmware ID: Reset Undefined | Medium |

Following below is a more detailed description of some of the representative types of POR events listed in Table 1, where it is understand that the types of POR events and associated severity ratings are not limited to these examples and may vary according to the particular IMD 10 being utilized and the particular POR events being monitored.

A Watchdog POR (Pulsed) is a method in which the hardware monitors firmware (CPU) operation. Normal operation requires firmware to restart a hardware controlled watchdog counter periodically. If the firmware does not restart the watchdog counter in the allowed time window or writes an incorrect restart sequence to the counter, the hardware generates a Pulsed Watchdog POR. By way of example, if a CPU of the IMD 10 becomes stuck in a loop, it would fail to restart the watchdog counter established by the hardware controller. An analogy would be a lock-up condition on a PC. After determining the CPU is not responding a hard reboot or POR is the only solution to regain control.

For an Atrial & Ventricle Rate Limit POR, a rate limit circuit for an ICD will provide an interval, which will be used to guard against runaway output pacing rates in the event of malfunction of the system crystal clock based timing. The interval is independent of the crystal clock (CPU clock) timing. This rate limit is suspended during pacing therapies, and any attempt to pace faster than the rate limit interval, without overriding the limit, will be inhibited, and the ICD shall POR. This may be assigned a medium POR Severity, where the save-to disk file should be reviewed by firmware engineering, but there is not a need for immediate replacement of the IMD.

Stack Overflow/Underflow POR is a common processor “house keeping” method of monitoring internal memory used by the CPU. The stack is memory space within the CPU allocated for software address pointers. These pointers are placed on the stack then removed from the stack when no longer valid. If the software timing is such that these pointers accumulate faster than removed the “keep-out” memory space, defined as the stack overflow address, will be reached. This will be detected by the processor and initiate a POR. Similarly if the CPU attempts to read an address from the stack prior to an address being placed on the stack, the processor will move to the stack underflow address to obtain the address for the operation, which will then initiate a POR. This may be assigned a medium POR severity, as the IMD 10 should be able to recover after the POR, and it should not occur again.

For a Memory Error POR, the IMD 10 performs tests on the RAM memory containing permanently programmed parameters to determine integrity. This test will check the static programmed parameters for certain operations periodically. For example, if a memory cell toggles value from logic 1 to logic 0 or visa-versa, within the periodic interval, this test will detect the value is invalid. The location of the invalid memory will be stored in reserved memory immediately before initiating POR.

For a Software Interrupt (SWI) Error POR, software interrupt instructions (SWI) are stored in the unused portions of the Read Only Memory (ROM). Should the CPU start to execute erroneously from within this area of ROM, a software interrupt instruction will initiate a POR.

For a VDD Glitch POR, VDD is the electrical term used to describe the integrated circuits power supply. The power supplied to VDD attempts to maintain a voltage level to ensure proper digital chip operation. A glitch detector circuit provides the means to detect transient conditions on the VDD supply source. It monitors the minimum voltage level and the time duration below the minimum level. A transthorasic shock may enter the IMD 10 circuitry from the lead system or capacitive coupled to the IMD 10 can result in transient conditions or momentary negative voltage excursion on the VDD supply. In this event the glitch detection circuit will generate a reset pulse to the CPU generating a POR. This has a high POR Severity, as there is an internal circuit problem.

For a VDD monitor POR (Level detector), this is similar to the VDD glitch detector only a longer duration of time below the minimum voltage level. In this event a reset pulse to the CPU will begin a POR.

For a Vreg Glitch POR, this is a hardware circuit similar to VDD that will detects transient conditions on the Voltage Regulated supply established for both analog and digital circuits. This regulated supply attempts to maintain non-interrupted power to the CPU. Just like VDD if the voltage drops below a minimum level the hardware will send a reset pulse to the CPU and initiate a POR. The Vreg Monitor POR is similar to the Vreg glitch detector, however this indicates the measured Vreg voltage was below the minimum voltage level for a longer duration. These both have high POR Severity, as there is an internal circuit problem.

For an Illegal Stop Key POR, if an incorrect value is written to the CPU shutdown register the IMD 10 will POR, where is considered a CPU error having a medium POR Severity that should be evaluated.

For a Critical RAM Parity Error POR, the cause is similar to a “Write to Locked RAM” POR, however the firmware will not attempt to perform a write to the address that contains the error—thus forgoing the Write to Locked RAM POR message. The critical RAM parity error can be verified by comparing the timestamp of the POR log to the parity error log. This has a low POR Severity and the device should be able to fully recover after the reset.

For a Write to Locked RAM POR, the processor attempts to write to an area of device RAM that is considered locked (programmed pacing parameters, detection, ram-ware, therapy, . . . ), the firmware will initiate a POR rather than over-write a critical parameter. This could be the result of a memory error if the write-to address was corrupted prior to making it to the CPU. *A special case exists for most parity errors that are detected in critical RAM. The parity error handling operation performs a write to the address that contains the error. Critical RAM is also considered locked RAM, meaning only special programming functions can write to that area of RAM. As a result, the firmware tries to write to a locked RAM address, and initiates a Write to Locked RAM POR. This POR is simply mislabeled, and can be verified as a critical RAM parity error by comparing the timestamp of the POR log to the parity error log. This has a low POR Severity and the device should be able to fully recover after the reset.

For a Write to ROM POR, if the CPU attempts to write to an area of the device memory that is defined as read-only, the IMD 10 will POR. This could be a CPU error, or a memory error if the write address was corrupted prior to making it to the CPU.

For a Write or Read to Illegal Address POR, if the processor attempts to read or write to an address that does not contain RAM, nor ROM, a POR is initiated. Similar error to the “Write to ROM” description.

An Illegal Pacing Mode POR will issue when the Brady engine new mode register is set to an undefined value. Similar to the “Write to ROM” error.

The NMI POR: (Non-Maskable Interrupt) is a firmware initiated reset from a self-monitored error. Unless the NMI is configured to execute RAMWARE code or to process a parity error, the detection of the NMI shall cause the firmware to initiate a reset of the IMD 10.

The Event Buffer Overflow POR is a firmware initiated reset. When an event interrupt occurs, the firmware shall compare the event pointer index with the event pointer index from the previous interrupt. If the difference between these two values is greater than 8, firmware shall log an event buffer overflow error and reset the IMD 10.

For a Parity Check POR, parity checking is a memory integrity check that can pin point the byte that contains the memory error in the lower portion of device RAM. Parity checking is a rather simple process that can detect single bit errors, by counting the number of bits that contain a “1” (binary) in an 8-bit byte. The parity bit is calculated by an odd count method. If there is an odd number of 1's in the 8-bits, the parity bit is set to “1”, and “0” on an even count. The parity bit is set on each byte when data is written. On every read of RAM data the parity will be checked by the hardware by reading bits 0-7 at the given address, calculate the parity as 0 or 1, and compare that to the stored parity bit at that address. If the comparison shows a difference, the hardware will cause a non-maskable interrupt (NMI) with an indication that a parity error has occurred. Firmware logs this parity error, storing the timestamp, memory address, and data associated with the parity error. If the error is detected in what is considered critical RAM (programmed pacing parameters, detection, ram-ware, therapy, . . . ) the firmware will initiate a Critical RAM Parity Error POR. This has a low POR Severity as memory errors will randomly occur in any electronic device with electronic memory. If this error occurs more than once to a device, it may suggest a chronic memory problem.

For an Illegal Op Code POR, hardware monitors the microprocessor for any attempt to perform an unrecognized, or illegal operation. When this occurs it will generate a special interrupt to cause the firmware to reset itself and the ICD. This is to prevent the firmware from getting stuck in a loop or getting lost in its operations.

For a CPU Error POR, the CPU used in the IMD 10 may include vectored interrupts, wherein all of the unimplemented vectored interrupts must have vectors to some interrupt service routine, since the possibility of a spurious interrupt still exists. All of these unimplemented vectored interrupts are handled by one service routine of a firmware initiated reset.

For a EEPROM POR, EEPROM memory integrity is tested as the device recovers from a POR. If there is a problem the device will not generate another patient alert, but simply record another POR reason in the POR log. This has a high POR Severity with recommended device replacement, as there is likely an internal circuit problem.

For a Firmware ID: Reset Undefined POR, if there is no reason for the IMD 10 reset identified in the hardware POR status registers, the firmware shall initiate a reset of the IMD 10.

The foregoing list of types of POR events that may be encountered by an IMD 10 are listed for the purposes of illustration only and it is understood that the system and methods described herein for POR detection and decoding can be implemented with any types of POR events that may be encountered by an IMD 10.

While the system and method have been described in terms of what are presently considered to be specific embodiments, the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

1. A method comprising:
receiving data communicated from a medical device related to operating parameters of the medical device;
performing a power-on-reset (POR) detection algorithm to analyze the received data to detect whether a power-on-reset (POR) event occurred on the medical device and, upon detecting the occurrence of a power-on-reset (POR) event, to assign a severity rating to detected power-on-reset (POR) event; and
generating an alert message containing an indication of the detection of the occurrence of a power-on-reset (POR) event in the medical device, information relating to the basis for the detected power-on-reset (POR) event, and a severity rating of the detected power-on-reset (POR) event.

2. The method of claim 1, wherein the generated alert message further contains information relating to the time when the power-on-reset (POR) event occurred in the medical device.

3. The method of claim 1, wherein the medical device is associated with a particular patient, the method further comprising communicating the generated alert message to at least one of the patient and a care provider for the patient.

4. The method of claim 1, wherein the data is received at a remote server that performs the power-on-reset (POR) detection algorithm.

5. The method of claim 4, further comprising storing historical information relating to detected power-on-reset (POR) events at the remote server.

6. The method of claim 5, further analyzing the stored historical information relating to detected power-on-reset (POR) events for diagnosing the operation of the medical device.

7. The method of claim 1, wherein the medical device is an implantable medical device (IMD).

8. The method of claim 8, further comprising:
communicating the data relating to the operating parameters of the IMD to an external device located in the proximity of a patient associated with the medical device; and
communicating the operating parameters data from the external device to a remote server where the power-on-reset (POR) detection algorithm is performed to analyze the operating parameters data.

9. A system for detecting power-on-reset (POR) events in a medical device, comprising:
a remote server including an input/output device for receiving data communicated from a medical device related to operating parameters of the medical device and a storage medium for storing the received data;
the remote server including a processor, a memory and a power-on-reset (POR) detection module stored in the memory, wherein the processor is configured to execute instructions contained within the stored power-on-reset (POR) detection module to execute a power-on-reset (POR) detection algorithm to perform the following steps:
analyze the received data to detect whether a power-on-reset (POR) event occurred on the medical device and, upon detecting the occurrence of a power-on-reset (POR) event, to assign a severity rating to the detected power-on-reset (POR) event; and
generate an alert message containing an indication of the detection of the occurrence of a power-on-reset (POR) event in the medical device, information relating to the basis for the detected power-on-reset (POR) event, and a severity rating of the detected power-on-reset (POR) event.

10. The system of claim 9, wherein the remote server is configured to include information relating to the time when the power-on-reset (POR) event occurred in the medical device in the generated alert message.

11. The system of claim 9, wherein the medical device is associated with a particular patient, wherein the remove server is further configured to communicate the generated alert message to at least one of the patient and a care provider for the patient.

12. The system of claim 9, wherein the power-on-reset (POR) detection algorithm performed on the remote server automatically generates the alert message upon the detection of a power-on-reset (POR) event.

13. The system of claim 9, wherein the remote server is further configured for storing historical information relating to detected power-on-reset (POR) events.

14. The system of claim 9, wherein the medical device is an implantable medical device (IMD).

15. The system of claim 14, further comprising:
an external device located in the proximity of a patient associated with the medical device, wherein the data relating to the operating parameters of the IMD is communicated to the external device which, in turn, communicates the operating parameters data on to the remote server where the power-on-reset (POR) detection algorithm is performed to analyze the operating parameters data.

16. A system comprising:
means for receiving data communicated from a medical device related to operating parameters of the medical device;
means for performing a power-on-reset (POR) detection algorithm to analyze the received data to detect whether a power-on-reset (POR) event occurred on the medical device and, upon detecting the occurrence of a power-on-reset (POR) event, to assign a severity rating to detected power-on-reset (POR) event; and
means for generating an alert message containing an indication of the detection of the occurrence of a power-on-reset (POR) event in the medical device, information relating to the basis for the detected power-on-reset (POR) event, and a severity rating of the detected power-on-reset (POR) event.

17. The system of claim 16, wherein the means for generating an alert message further includes information relating to the time when the power-on-reset (POR) event occurred in the medical device in the alert message.

18. The system of claim 16, wherein the medical device is associated with a particular patient, the system further comprising means for communicating the generated alert message to at least one of the patient and a care provider for the patient.

19. The system of claim 16, further comprising means for storing historical information relating to detected power-on-reset (POR) events at the remote server and means analyzing the stored historical information relating to detected power-on-reset (POR) events for diagnosing the operation of the medical device.
